(19) United States
(12) Patent Application Publication
(10) Pub. No.: US 2023/0363919 A1
(43) Pub. Date: Nov. 16, 2023

(54) METHOD FOR PRODUCING A SLIDING SURFACE ELEMENT, SLIDING SURFACE ELEMENT AND KNEE JOINT ENDOPROSTHESIS

(71) Applicant: Aesculap AG, Tuttlingen (DE)

(72) Inventors: Berna Richter, Muehlheim (DE); Thomas Grupp, Denkingen (DE); Marie Anne Mulliez, Tuttlingen (DE); Jens Schwiesau, Tuttlingen (DE); Arthur Bollinger, Tuttlingen (DE)

(21) Appl. No.: 18/355,693

(22) Filed: Jul. 20, 2023

Related U.S. Application Data
(63) Continuation of application No. PCT/EP2022/051594, filed on Jan. 25, 2022.

(30) Foreign Application Priority Data
Feb. 9, 2021 (DE) 10 2021 103 016.1

(51) Int. Cl. A61F 2/38; A61F 2/30; A61L 27/18

(52) U.S. Cl. CPC A61F 2/3868; A61F 2/3094; A61L 27/18; A61L 2430/24

(57) ABSTRACT

A method for producing a sliding surface element on the basis of UHMWPE for a joint endoprosthesis includes mixing UHMWPE in powder form with 0.09 to 0.11% by weight of an antioxidant, compacting the UHMWPE mixed with the antioxidant into a molded body, manufacturing one or more sliding surface elements from the molded body by material-removing machining, and irradiating the sliding surface element by gamma radiation or X-ray radiation with a radiation dose of 25 to 45 kGy in order to crosslink the UHMWPE. The method can be practiced without thermal post-treatment of the irradiated sliding surface element.

### CROSS-REFERENCE TO RELATED APPLICATIONS

This application is a continuation of International Application No. PCT/EP2022/051594 filed on Jan. 25, 2022 and claims priority to German Application No. 10 2021 103 016.1 of Feb. 9, 2021. The contents of International Application No. PCT/EP2022/051594 and German Application No. 10 2021 103 016.1 are incorporated herein by reference in their entireties and for all purposes.

### FIELD

The present disclosure relates to methods for producing sliding surface elements in general, and more specifically to a method for producing a sliding surface element on the basis of UHMWPE for a joint endoprosthesis, in particular for a knee joint endoprosthesis.

The present disclosure also relates to sliding surface elements in general, and more specifically to a sliding surface element for a joint endoprosthesis, which is produced according to this method.

Finally, the present disclosure relates to knee joint endoprostheses in general, and more specifically to a knee joint endoprosthesis with a femoral component and a tibial component, wherein a sliding surface element of that kind is fixed to the tibial component.

### BACKGROUND

Joint endoprostheses have been known for a long time and comprise sliding surface elements made of ultra-high molecular weight polyethylene (UHMWPE). Here, the sliding surface element made of UHMWPE typically cooperates with a corresponding element made of a metallic material, whereby this material combination results in a low coefficient of friction. In a hip joint prosthesis, the sliding surface element made of UHMWPE is typically formed as an inlay of the hip head implant, while in a knee joint prosthesis it is typically fixed to the tibial component and is also referred to as a meniscus element. A sliding surface made of UHMWPE can also be used for a kneecap prosthesis (patella prosthesis).

The sliding surface element must meet certain requirements with regard to its mechanical properties, wear resistance and aging resistance, whereby the material properties of the UHMWPE required for this purpose are partly in opposition. To increase wear resistance, UHMWPE can be crosslinked with ionizing radiation, as described in EP 0 995 450 A1, for example. At the same time, however, crosslinking reduces the ductility of the material, which increases the risk of structural material fatigue, delamination and, as the case may be, failure of the sliding surface element.

Since free radicals are formed during radiation crosslinking of UHMWPE and remain in part in the material, this tends to lead to a poorer aging resistance due to oxidative processes. This problem can be counteracted at least partially by the addition of antioxidants such as, e.g., a-tocopherol (vitamin E), wherein the antioxidant is either added to the UHMWPE before the production of a molded body or is introduced into the molded body by diffusion only after crosslinking (see, for example, EP 3 111 895 A1). In view of the fact that younger patients are increasingly being treated with joint endoprostheses, and that at the same time life expectancy is increasing and patients remain active at an older age, the aging resistance of UHMWPE in joint endoprostheses is becoming increasingly important.

The requirements for the property profile of the UHMWPE also differ depending on the type of joint endoprosthesis. In a hip joint prosthesis, the cooperating sliding surfaces are substantially congruent (hemispherical), so that the occurring forces can be distributed more or less evenly over the entire contact surface. Therefore, the ductility of the material does not play a significant role here. The conditions for a knee joint prosthesis are quite different, where the cooperating sliding surfaces are far less congruent, in order to be able to achieve the desired freedom of movement of the joint (in the sagittal plane and in the frontal plane). In contrast to the hip joint, sliding and rolling movements take place simultaneously, whereby punctual force effects and stress peaks occur, which are up to a factor of 10 higher than with the hip. For sliding surface elements made of highly crosslinked UHMWPE with too low ductility, it has been shown that this can lead to cracks, delamination, and ultimately to fracture and failure of the sliding surface element.

### SUMMARY

In a first aspect of the present disclosure, a method is provided for producing a sliding surface element on the basis of UHMWPE for a joint endoprosthesis, comprising the steps:
mixing UHMWPE in powder form with 0.09 to 0.11% by weight of an antioxidant;
compacting the UHMWPE mixed with the antioxidant into a molded body;
manufacturing one or more sliding surface elements from the molded body by material-removing machining; and
irradiating the sliding surface element by gamma radiation or X-ray radiation with a radiation dose of 25 to 45 kGy in order to crosslink the UHMWPE,
wherein the method comprises no thermal post-treatment of the irradiated sliding surface element.

In a second aspect of the present disclosure, a sliding surface element on the basis of UHMWPE for a joint endoprosthesis is provided, which sliding surface element is produced in accordance with a method of the first aspect of the present disclosure.

In a third aspect of the present disclosure, a knee-joint endoprosthesis is provided, comprising a femoral component and a tibial component, wherein a sliding surface element in accordance with the second aspect of the present disclosure is fixed to the tibial component and cooperates with the femoral component.

### BRIEF DESCRIPTION OF THE DRAWING FIGURES

The foregoing summary and following description may be better understood in conjunction with the drawing figures, of which:

FIG. 1 shows a radar chart of the property profile of various UHMWPE materials; and

FIG. 2 shows a diagram of the compressive stress curve in the case of a knee joint endoprosthesis with a sliding surface element in accordance with the present disclosure.

### DETAILED DESCRIPTION

Although the present disclosure is illustrated and described herein with reference to specific embodiments, the present disclosure is not intended to be limited to the details shown. Rather, various modifications may be made in the details within the scope and range of equivalents and without departing from the present disclosure.

The present disclosure relates to a method for producing a sliding surface element on the basis of UHMWPE for a joint endoprosthesis, comprising the steps:
mixing UHMWPE in powder form with 0.09 to 0.11% by weight of an antioxidant;
compacting the UHMWPE mixed with the antioxidant into a molded body;
manufacturing one or more sliding surface elements from the molded body by material-removing machining; and
irradiating the sliding surface element by gamma radiation or X-ray radiation with a radiation dose of 25 to 45 kGy in order to crosslink the UHMWPE,
wherein the method comprises no thermal post-treatment of the irradiated sliding surface element.

Surprisingly, it has been shown that the combination of the above method parameters makes it possible to produce a sliding surface element with a high wear resistance, aging resistance, mechanical strength, and ductility. In particular, the only moderate crosslinking of the UHMWPE with a relatively low radiation dose using gamma radiation or X-ray radiation is decisive, which have a significantly lower intensity and absorption compared to beta radiation. Furthermore, in the method in accordance with the present disclosure, no thermal post-treatment of the irradiated sliding surface element is performed, which can lead to unfavorable material changes and reduction of strength. By eliminating such post-treatment, the method is also more cost-effective.

The UHMWPE used within the scope of the present disclosure typically has a molecular weight in the range of 5.106 to 107 g/mol (determined from the intrinsic viscosity) and a density in the range of 0.92 to 0.95 g/cm³. A suitable UHMWPE in powder form is available, e.g., from Ticona GmbH under the name GUR 1020, or as a mixture with 0.1% by weight of a-Tocopherol as antioxidant under the name GUR 1020-E.

Various antioxidizing agents that are also approved for use in the medical field may be used as an antioxidant with which the UHMWPE is mixed. The antioxidant is preferably selected from tocopherols, tocotrienols, ascorbic acid, polyphenolic antioxidants like, e.g., flavonoids, butylhydroxytoluol (BTH), and butylhydroxyanisole (BTA).

In a preferred embodiment of the present disclosure, the antioxidant is a-tocopherol. This is also referred to as vitamin E, the term vitamin E comprising in a broader sense all tocopherols, tocotrienols, and further fat-soluble antioxidants.

The compacting of the UHMWPE into a molded body is typically carried out by means of compression molding or RAM extrusion, preferably up to a density of the molded body of 0.92 g/cm³ or more, i.e. substantially up to reaching the theoretical density of the UHMWPE. The compacted molded body typically has a dimension in each spatial direction of 50 mm or more, preferably of 100 mm or more, so that from this blank a plurality of sliding surface elements can be produced by material-removing machining, in particular by means of milling.

In accordance with the present disclosure, the irradiation of the finished sliding surface element with its final geometry is then performed. This ensures uniform crosslinking, in particular in the surface region of the sliding surface element, in contrast to such methods in which first a molded body is irradiated as a blank and then the production of the respective components takes place.

The radiation dose is preferably 27 to 33 kGy, and further preferably about 30 kGy. It has been shown that with a radiation dose of this magnitude an optimal balance can be achieved between the opposing requirements, in particular the wear resistance and the ductility of the sliding surface element.

The irradiation of the sliding surface element with gamma radiation is preferably carried out over a period of 4 to 6 h. At a preferred radiation dose of 30 kGy, this corresponds to a dose rate in the range of 5 to 7.5 kGy/h.

The irradiation of the sliding surface element with X-ray radiation is preferably performed over a period of 1 to 10 minutes. At a preferred radiation dose of 30 kGy, this corresponds to a dose rate in the range of 0.05 to 0.5 kGy/s.

It is particularly favorable if the sliding surface element is packaged with a plastic film in a germ-tight manner before irradiation. In addition to crosslinking, the gamma radiation also sterilizes the sliding surface element in the hermetic packaging.

Irradiation of the sliding surface element is preferably performed in a receiving room, which has a total filling of up to 250 kg per m³.

Further, the present disclosure relates to a sliding surface element on the basis of UHMWPE for a joint endoprosthesis that is produced according to a method in accordance with the present disclosure.

The particular advantages and preferred embodiments of the sliding surface element in accordance with the present disclosure were already discussed in connection with the method in accordance with the present disclosure.

It is particularly advantageous if the sliding surface element in accordance with the present disclosure is a sliding surface element for a knee joint endoprosthesis, in particular for a tibial component or patella component of the knee joint endoprosthesis. In this context, the sliding surface element can also be called a meniscus element.

The advantageous mechanical properties of the sliding surface element, which result from the application of the manufacturing method in accordance with the present disclosure and which make the use in a knee joint endoprosthesis possible, can be characterized in particular by one or more of the following parameters:

The sliding surface element preferably has an elongation at break of 400% or more, further preferably of 450% or more.

The sliding surface element preferably has a tensile strength of 50 MPa or more, further preferably of 55 MPa.

The sliding surface element preferably has a yield stress of 20 MPa or more, in particular of 22 MPa or more.

The sliding surface element preferably has an Izod impact strength of 100 kJ/m² or more, further preferably of 110 kJ/m² or more.

The swelling ratio can be used as a measure of the degree of crosslinking of UHMWPE, the swelling ratio decreasing with increasing crosslinking. The sliding surface element in accordance with the present disclosure preferably has a swelling ratio of over 4, resulting from the only moderate crosslinking in the method in accordance with the present disclosure. Here, the swelling ratio is determined in accordance with the ASTM F 2214:2016 standard (determination of the change in volume of the crosslinked UHMWPE due to swelling in o-xylene).

Further, the present disclosure relates to a knee joint endoprosthesis comprising a femoral component and a tibial component, wherein a sliding surface element in accordance with the present disclosure is fixed to the tibial component and cooperates with the femoral component. Here, the radii ratios of the surface regions of the femoral component and the sliding surface element, which come into contact with one another over the range of motion of the knee joint endoprosthesis, are in the range of 1 to 7.

The wide range of radii ratios in the knee joint endoprosthesis in accordance with the present disclosure illustrates the low congruency (e.g. in comparison to a hip joint prosthesis) between the femoral component and the sliding surface element, which is required in a knee joint prosthesis in order to achieve the desired freedom of movement during the manipulation of the joint in the sagittal plane and in the frontal plane.

The compressive stresses acting on the sliding surface element in the case of the knee joint endoprosthesis in accordance with the present disclosure, in particular point loads, are typically up to 25 MPa. Due to the mechanical properties of the sliding surface element in accordance with the present disclosure, these values are not critical.

### Property Profile of Crosslinked UHMWPE

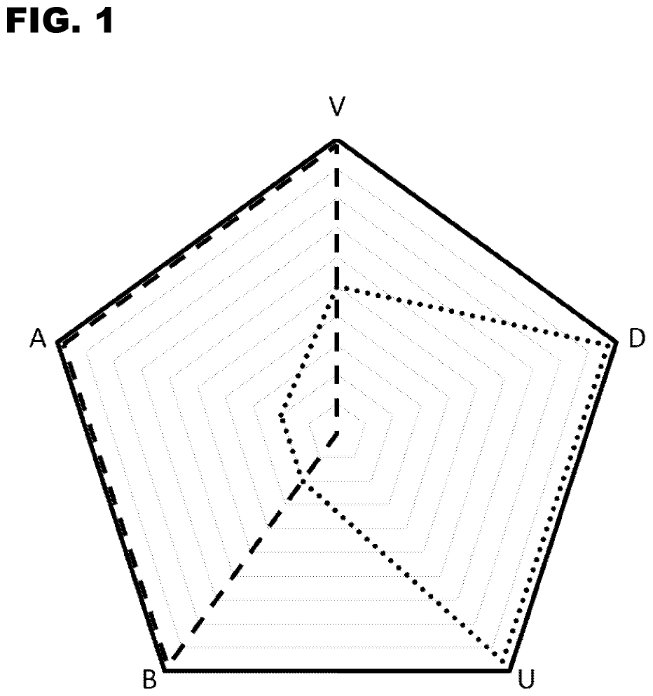

The radar chart in FIG. 1 schematically shows the typical property profile of the moderately crosslinked UHMWPE of the sliding surface element in accordance with the present disclosure (solid line), a highly crosslinked UHMWPE with the addition of vitamin E (dashed line), and a standard UHMWPE without the addition of vitamin E (dotted line).

The five axes of the diagram represent the following properties of the materials:
V: Wear resistance
D: Ductility
U: Insensitivity to stress peaks
B: Freedom of movement (due to low congruency in the joint)
A: Aging resistance

The diagram shows schematically that all required properties can be achieved to a high extent with the sliding surface element in accordance with the present disclosure, whereas this is not possible with the UHMWPE materials of the prior art, in particular due to the opposing effects of a high degree of crosslinking. Although this enables high wear resistance and aging resistance, it also reduces the ductility of the material and makes it sensitive to stress peaks. In addition, the sliding surface element in accordance with the present disclosure enables a particularly high kinematic freedom of movement of the joint endoprosthesis by withstanding the high point loads that occur with less congruent joint partners.

### Example

To produce a sliding surface element in accordance with the present disclosure, powdered UHMWPE (GUR 1020, Ticona GmbH) was homogeneously mixed with 0.1% by weight of vitamin E (a-tocopherol) and pressed into a plate. A sliding surface element for a tibial component of a knee joint endoprosthesis was milled out of this blank. After packaging in film, the sliding surface element was irradiated with gamma radiation for about five hours in order to crosslink and simultaneously sterilize the UHMWPE. Here, the radiation dose was 30±3 kGy.

In the following table, the relevant mechanical properties of the sliding surface element produced in accordance with the present disclosure are given, as well as the corresponding values (from the literature) for commercially available, crosslinked UHMWPE materials for comparison:

| | Elongation | Tensile | Yield | Izod impact |
|---|---|---|---|---|
| | at break | strength | stress | strength |
| Present disclosure | 457 ± | 59.7 ± | 22.4 ± | 119.4 ± |
| | 9% | 2.9 MPa | 0.1 MPa | 2.2 kJ/m² |
| XLPE | 300 ± | 56 ± | 20 ± | N/A |
| (Smith & Nephew) | 20% | 7.1 MPa | 1.3 MPa | |
| Marathon | 290 ± | 56 ± | 21 ± | N/A |
| (DePuy/J and J) | 14% | 5.7 MPa | 1.5 MPa | |
| Crossfire | 280 ± | 48 ± | 24 ± | N/A |
| (Stryker Howmedica) | 37% | 7.2 MPa | 1.3 MPa | |

The comparison shows that the sliding surface element in accordance with the present disclosure is significantly superior to the prior art, especially in the case of elongation at break. The tensile strength of the UHMWPE in accordance with the present disclosure is also higher than for all three known materials.

To determine the loads in a knee joint endoprosthesis, the sliding surface element in accordance with the present disclosure was combined as a meniscus component with a femoral component, wherein the radii ratios of the surface regions that come into contact with one another over the range of motion of the joint are in the range of 1 to 7.

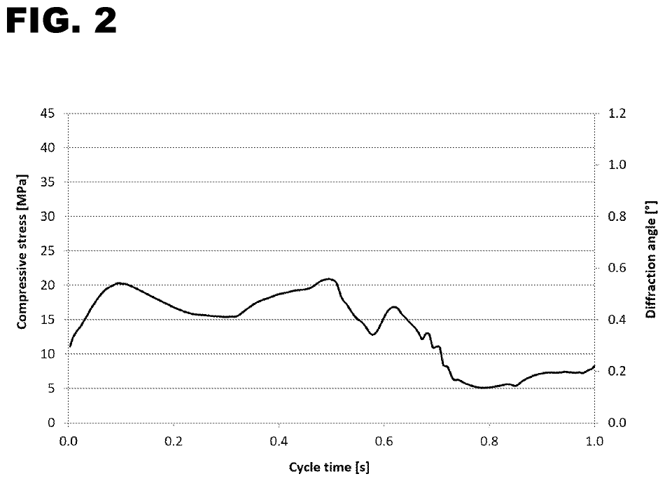

The profile of the compressive stress between the sliding surface element and the femoral component over a cycle time of one second is shown in FIG. 2. The values are consistently below 25 MPa and are not critical with regard to the mechanical properties of the sliding surface element in accordance with the present disclosure.

What is claimed is:

1. A method for producing a sliding surface element based on UHMWPE for a joint endoprosthesis, the method comprising the steps of:
mixing UHMWPE in powder form with 0.09 to 0.11% by weight of an antioxidant;
compacting the UHMWPE mixed with the antioxidant into a molded body;
manufacturing one or more sliding surface elements from the molded body by material-removing machining; and
irradiating the sliding surface element by gamma radiation or X-ray radiation with a radiation dose of 25 to 45 kGy in order to crosslink the UHMWPE,
wherein the method comprises no thermal post-treatment of the irradiated sliding surface element.

2. The method according to claim 1, wherein the UHMWPE has a molecular weight of 5*106 g/mol to 107 g/mol and a density of 0.92 g/cm³ to 0.95 g/cm³.

3. The method according to claim 1, wherein the antioxidant is selected from the group consisting of tocopherols, tocotrienols, ascorbic acid, polyphenolic antioxidants, butylhydroxytoluol, and butylhydroxyanisole.

4. The method according to claim 3, wherein the antioxidant is a-tocopherol.

5. The method according to claim 1, wherein the UHMWPE mixed with the antioxidant is compressed by compression molding or RAM extrusion.

6. The method according to claim 1, wherein the radiation dose is 27 to 33 kGy.

7. The method according to claim 1, wherein irradiation of the sliding surface element is performed with gamma radiation over a period of 4 to 6 h or with X-ray radiation over a period of 1 to 10 min.

8. The method according to claim 1, wherein the sliding surface element is packaged in a plastic film in a germ-tight manner before irradiation.

9. The method according to claim 1, wherein the irradiation of the sliding surface element is performed in a receiving space that has a total filling of up to 250 kg per m³.

10. A sliding surface element based on UHMWPE for a joint endoprosthesis, which is produced according to the method according to claim 1.

11. The sliding surface element according to claim 10, wherein the sliding surface element is a sliding surface element for a knee joint endoprosthesis.

12. The sliding surface element according to claim 10, wherein the sliding surface element has an elongation at break of 400% or more.

13. The sliding surface element according to claim 10, wherein the sliding surface element has a tensile strength of 50 MPa or more.

14. The sliding surface element according to claim 10, wherein the sliding surface element has a yield stress of 20 MPa or more.

15. The sliding surface element according to claim 10, wherein the sliding surface element has an Izod impact strength of 100 kJ/m² or more.

16. The sliding surface element according to claim 10, wherein the sliding surface element has a swelling ratio of over 4, determined in accordance with ASTM F 2214:2016.

17. A knee joint endoprosthesis comprising:
the sliding surface element according to claim 10;
a femoral component; and
a tibial component,
wherein a sliding surface element is fixed to the tibial component and cooperates with the femoral component, and
wherein the radii ratios of surface regions of the femoral component and the sliding surface element that come into contact with one another over the range of motion of the knee joint endoprosthesis are in the range of 1 to 7.
